(19) United States
(12) Patent Application Publication
(10) Pub. No.: US 2004/0253138 A1
(43) Pub. Date: Dec. 16, 2004

(54) Plasmon enhanced body treatment and bacterial management

(75) Inventor: Henryk Malak, Ellicott City, MD (US)

Correspondence Address: Henryk Malak, 8444 High Ridge Road, Ellicott City, MD, 21043, US

(73) Assignee: American Environmental Systems, Inc.

(21) Appl. No.: 10/689,965

(22) Filed: Oct. 22, 2003

Related U.S. Application Data
(60) Provisional application No. 60/478,260, filed on Jun. 16, 2003.

(51) Int. Cl. A61L 2/08

(52) U.S. Cl. 422/22; 422/28

(57) ABSTRACT

Methods and compositions of a surface plasmon resonance enhanced body treatment and bacterial management are described. Under the enhanced interaction of surface plasmon resonance and a metal nanoparticle with a nearby biological substance, the biological substance is biochemically and/or biophysically modified or destroyed. The methods and compositions use electromagnetic radiation at a single wavelength or plurality wavelengths of 200 nm to 10,000 nm, metal nanoparticles in size 1 nm to 20,000 nm, inorganic or organic chemical agents, and one-photon or multi-photon modes of electromagnetic radiation for surface plasmon resonance generation.

### CROSS REFERENCE TO RELATED APPLICATIONS

This application is related to U.S. Provisional Patent Application No. entitled “Plasmon Enhanced Body and Bacterial Management” filed Jun. 16, 2003, which is herein incorporated by reference.

### REFERENCES CITED

The following are patents found that may be associated with this information.

U.S. Patent Documents

| | | |
|---|---|---|
| U.S. Pat. No. 5,866,433 | Feb. 2, 1999 | Schalkhammer, et al. |
| U.S. Pat. No. RE37,412 | Oct. 16, 2001 | Schalkhammer, et al. |
| U.S. Pat. No. 6,071,541 | Jun. 6, 2000 | Murad |
| U.S. Pat. No. 6,383,523 | May 7, 2002 | Murad |
| U.S. Pat. No. 6,379,712 | Apr. 30, 2002 | Yan et al. |
| U.S. Pat. No. 5,785,972 | Jul. 28, 1998 | Tyler |
| U.S. Pat. No. 5,709,870 | Jan. 20, 1998 | Yoshimura, et al. |
| U.S. Pat. No. 4,203,765 | May 20, 1980 | Claeys, et al. |
| U.S. Pat. No. 4,828,832 | May 9, 1989 | De Cuellar, et al. |
| U.S. Pat. No. 5,824,267 | Oct. 20, 1998 | Kawasumi, et al. |
| U.S. Pat. No. 6,358,516 | Mar. 19, 2002 | Harod, et al. |

Foreign Patent Documents

| | |
|---|---|
| 2,189,394 | Great Britain |
| 87100231A | China |
| 3-136649 | Japan |
| 54-151669 | Japan |

### STATEMENT REGARDING FEDERALLY SPONSORED RESEARCH

There is NO claim for federal support in research or development of this product.

### BACKGROUND OF THE INVENTION

This invention relates to methods and compositions of a surface plasmon resonance enhanced body treatment and bacterial management. Under the enhanced interaction of surface plasmon resonance and a metal nanoparticle with a nearby biological substance, the biological substance is biochemically and/or biophysically modified or destroyed. The presence of electromagnetic radiation and chemical agents additionally enhance the changes in biological substances.

### DESCRIPTION OF THE PRIOR ART

U.S. Pat. No. 5,866,433 discloses an optochemical fluorescence sensor with a biorecognitive layer for measuring the concentration of one or more analytes in a sample is provided with at least one metal island layer that is applied on a sensor substrate. The islands of the island layer are in the form of electrically-conductive material and have a diameter of less than 300 nm, the biorecognitive layer being directly applied on the island layer or bound via a spacer film. In addition, an analyte-specific fluorescent compound is provided which may be added to the sample or is provided in the sensor itself. The biorecognitive layer can bind the analyte to be measured directly or by means of analyte-binding molecules, the originally low quantum yield of the fluorescent compound increasing strongly in the vicinity of the island layer.

U.S. Pat. No. RE37,412 discloses an optochemical sensor for measuring concentrations of analytes is provided with a reactive matrix preferably made of polymeric material capable of swelling. Further provided are a mirror layer and a layer of a plurality of discrete islands that are electrically conductive, between which layers the reactive matrix is positioned, the diameter of the islands being smaller than the wavelength of the light employed for monitoring and evaluation.

U.S. Pat. No. 4,203,765 discloses an aqueous acidic etch-bleach solution of hydrogen peroxide, iron ions, and inorganic anions that form a silver salt, such that in the dissolved state the solution contains citric acid and a polymer of alkylene oxide units for stabilization of the hydrogen peroxide.

Great Britain Application No. 2,189,394 A discloses a concentrate that can be mixed with hydrogen peroxide to become an effective disinfectant for water, foodstuff, animal feeds, equipment, packages, and the like. The concentrate includes an inorganic acid with a pH less than 1.6, a silver compound or colloidal silver, an organic acid stabilizer such as tartaric, lactic, salicylic, or citric acid, and optionally gelatin.

U.S. Pat. No. 6,379,712 This invention relates to nanosilver-containing antibacterial and antifingal granules (“NAGs”). The nanosilver particles are about 1-100 nm in diameter. Each of the nanosilver particles contain a metallic silver core which is surrounded by silver oxide. The present invention also provides a process for making the NAGs. The NAGs can be used in a variety of healthcare and industrial products. Examples of the healthcare products include, but are not limited to, ointments or lotions to treat skin trauma, soaking solutions or cleansing solutions for dental or women hygiene, medications for treating gastrointestinal bacteria infections, sexual related diseases, and eye diseases. Examples of industrial products include, but are not limited to, food preservatives, water disinfectants, paper disinfectants, construction filling materials (to prevent mold formation).

U.S. Pat. No. 5,785,972 discloses a therapeutically active composition comprising a solution of colloidal silver, helichrysum angustifolium or helichrysum italicum oil, and raw honey emulsified with water soluble lecithin. However, the contact between microbial cells and silver ions is not ensured as the silver ions quickly become eluted in the solution Silver ions in solution are difficult to handle and therefore of limited use.

U.S. Pat. No. 5,709,870 discloses a silver-containing antimicrobial agent comprising a silver salt of carboxymethylcellulose and having a degree of substitution of carboxymethyl group of not less than 0.4.

Chinese Patent No. 87100231A discloses an antibacterial dressing made from nitrilon crosslinked with copper salts in alkaline medium The resulted cloth shows antibacterial activity on ten (10) bacteria including Staphylococcus aureus (MRSA).

Japanese Patent No. 3-136649 discloses an anti-bacterial cloth used for washing breasts of milk cow. The Ag.sup.+ions in AgNO.sub.3 were crosslinked with polyacrylonitrile and it had anti-bacterial activity on six (6) bacteria including Streptococcus and Staphylococcus.

Japanese Patent No. 54-151669 discloses a fiber treated with a solution of a compound of copper and silver. The solution is evenly distributed on the fiber. The fiber is used as an anti-bacterial lining inside boots, shoes, and pants.

U.S. Pat. No. 4,828,832 discloses a composition for treating skin lesions which is made up of metallic silver particles having a diameter of 1 to 10 micron and an optional oxidizing agent randomly disbursed within a carrier of inert filler such as kaolin or talc.

U.S. Pat. No. 5,824,267 discloses a plastic material having a bactericidal surface on which a number of ceramic or base metal particles of a mean diameter of 0.01 to 0.5 micron are embedded under the condition that a portion of each particle is exposed over the surface, and the ceramic or base metal particles have bactericidal metal particles of mean diameter of 0.0001 to 0.1 micron dispersively fixed thereon.

U.S. Pat. No. 6,358,516 discloses a skin care system that cleanses, therapeutically conditions, and provides additional beneficial treatment to the skin in a simple, one-step application that air dries quickly. The system is implemented as a skin care kit in the form of a container with a plurality of pre-moistened soft cloths therein. The cloths are impregnated with a treatment composition that contains a plurality of ingredients selected from the following groups: (a) surfactants, (b) anti-inflammatory agents, (c) non-foaming agents, (d) cell-growth-promoting agents, (e) immune system-enhancing agents, (f) antimicrobial agents, (g) absorption facilitating agents, (h) humectants and emollients, (i) free radical-scavenging agents, (j) healing promoting agents, and (optionally) preservatives and fragrances. In use, the cloths gently cleanse the skin, trap and carry away dirt and soil, and deposit beneficial ingredients that coat and are absorbed into the skin. The system is portable, disposable, easily stored, and can be partially used and resealed for further use.

Rami Pedahzur et al., The interaction of silver ions and hydrogen peroxide in the inactivation of E. coli: a preliminary evaluation of a new long acting residual drinking water disinfectant, Water Science and Technology Vol 31 No 5-6 pp 123-129 (1995) discloses study of a performance evaluation of the combined disinfectant for drinking water applications. The major advantages of such combined disinfectant include, low toxicity of its components, long lasting residual effect and low disinfection by product formation. Specific strains of E. coli (E. coli-B (SR-9) and E. coli K-12) were used in this study as target microorganisms and the separate and combined inactivation efficiencies of silver and hydrogen peroxide were evaluated at different concentrations and exposure durations. Both, silver and hydrogen peroxide exhibited a significant inactivation performance even at concentrations that do not pose any health risk according to the EEC, WHO and the USEPA (the USEPA Maximum Contaminant Level (MCL) of silver is 90 ppb, and currently, there is no MCL for hydrogen peroxide but it is approved as a food additive in the USA). Combinations of 1:1000 silver:hydrogen peroxide (w) exhibited higher inactivation performance as compared with each of the disinfectants alone and in some cases a synergistic effect was observed, i.e., the combined disinfectant exhibited higher inactivation performance than the sum of the inactivation levels of the separate disinfectants. Thus, for example, one hour exposure to 30 ppb silver, 30 ppm hydrogen peroxide and their combination yielded 2.87, 0.65 and 5 logs of inactivation respectively. While the rate of inactivation shown by this combined disinfectant, now available commercially in a stabilized formulation is relatively slow, it may well hold promise as a secondary disinfectant providing long lasting residuals and biofilm control required for distribution systems. Its disinfection action may be similar to chloramines, the use of which has been recently outlawed in France and in Germany and which are now under careful scrutiny in other countries due to the formation of undesirable by-products.

### SUMMARY OF THE INVENTION

The objective of the invention is to provide methods and compositions of a surface plasmon resonance enhanced interaction of metal nanoparticles with biological substances. This objective is achieved by applying surface plasmon resonance of metal nanoparticles to enhance the interactions of metal nanoparticles with nearby biological substances in the presence of nearby chemical agents and electromagnetic radiation.

The invention expands the analytical capacity of conventional interaction of colloidal metals with biological substances in antiseptic technology and human/animal body treatment. The invention describes methods of the enhanced interaction of metal nanoparticles with biological substances induced by nonlinear generated surface plasmon resonance, multiwavelength and multimode electromagnetic radiation, and chemical agents.

### BRIEF DESCRIPTION OF THE DRAWINGS

FIG. 1. Schematic diagam of a surface plasmon resonance enhanced interaction of a metal nanoparticle with a nearby biological substance in the presence of a nearby chemical agent and electromagnetic radiation. Electromagnetic radiation in the invention can be applied at multiple wavelengths, one of wavelengths may generate surface plasmon resonance and other wavelengths may interact with the biological substance and the chemical agent.

FIG. 2. Description of a metal nanoparticle coated with biorecognitive polymer containing a drug for a localized surface plasmon resonance enhanced body treatment.

FIG. 3. Schematic diagram of a surface plasmon resonance enhanced interaction of biorecognitive polymer coated metal nanoparticles with bacteria in the presence of chemical agents and multiple wavelengths of electromagnetic radiation. In that case, bacteria in an aerosol form is captured by biorecognitive site and destroyed by surface plasmon resonance.

FIG. 4. An example of an experimental set up for capture and killing bacteria in bioaerosol is showed here, where surface plasmon resonance is generated by two-photon excitation and amino acids in bacteria are excited by 266 nm, a fourth harmonic of a Nd:Yag laser.

### DETAILED DESCRIPTION OF THE INVENTION

The invention provides a novel methodology that overcomes limitations of conventional methods of using colloidal metals in bacterial management and body treatment.

In the absence of surface plasmon resonance, current use of colloidal metals is restricted by the need of high doses of colloidal metals to be effective in body treatment or bacterial killing. Biological substances are modified or destroyed by colloidal metals mainly in direct contact with them. If metal nanoparticles in colloidal metals are coated with a dielectrical layer, interactions between colloidal metals and biological substances are negligible.

The invention relates to the scientific reports of enhanced interaction between metal nanoparticles with molecules in the presence of surface plasmon resonance (M. Kerker, “Optics of colloid silver”, J. Colloid Interface Sci. 105, 298 (1985); Lakowicz et al, “Intrinsic fluorescence from DNA can be enhanced by metallic particles”, Biochem. Biophys. Res. Comm. 286, 875 (2001); Gryczynski et al., “Multiphoton excitation of fluorescence near metallic particles: enhanced and localized excitation”, J. Phys. Chem. B, 106, 2191 (2002)). In these reports, researchers use fluorophores (mostly organic laser dyes) to visualize or test the surface plasmon resonance enhanced interaction. Their studies show that the fluorescence intensity of the fluorophores can be enhanced by a factor as high as ˜10⁴ with one-photon mode excitation or ˜10⁸ with two-photon mode of excitation, and this enhancement occurs at distances up to 500 nm from metal nanoparticles (M. Moskovits: Rev. Mod. Phys. 57, 783 (1985); T. L. Haslett, L. Tay, M. Moskovits: J. Chem. Phys. 113, 1641 (2000), and references therein K. Kneipp, Y. Wang, H. Kneipp, L. T. Perelman, I. Itzkan, R. R. Dasari M. S. Feld: Phys. Rev. Lett. 78, 1667 (1997); Gryczynski et al., “Multiphoton excitation of fluorescence near metallic particles: enhanced and localized excitation”, J. Phys. Chem. B, 106, 2191 (2002)). However, observed surface plasmon resonance enhanced interaction of fluorophore with metal nanoparticle was associated with intense fluorophore photobleaching (Ditlbacher H. et al., Appl. Phys. B 73, 373-377 (2001)).

This invention expands the above scientific findings to a new method of a surface plasmon resonance enhanced interaction of metal nanoparticles with biological substances that leads to biochemical/biophysical modifications or destruction of biological substances. Biological substances considered in this invention are: a biomolecule, bacteria, living tissue, cells, virus, human body, animal body, and other living biological species.

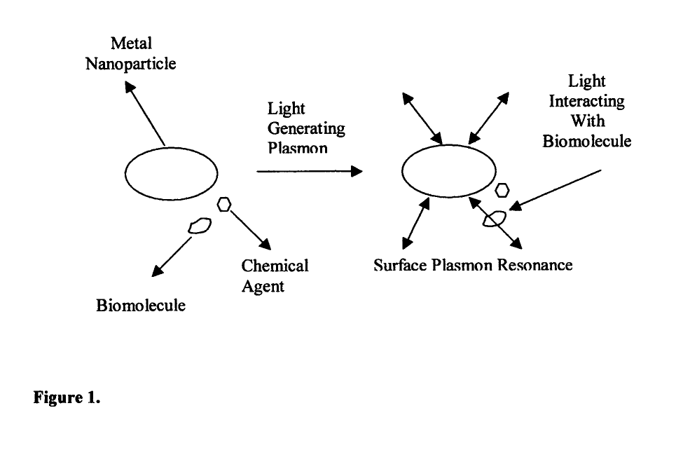

In the presence of surface plasmon resonance, metal nanoparticles interact with biological substances not only in direct contact with them, but also at a distance from metal nanoparticles, where intense electromagnetic fields exist. Under such enhanced interaction, biological substances may change their biological functionality or be destroyed. The changes in biological substances or their destruction induced by surface plasmon resonance and electromagnetic radiation can be used for more effective body treatment, bacterial killing, and other applications. An additional innovation of the method is the possibility of using plurality wavelengths of electromagnetic radiation, where for example one of the wavelengths is used for surface plasmon resonance generation and the other wavelengths are used for interaction of electromagnetic radiation with biological substances and chemical agents (FIG. 1). The enhanced interaction will have positive impact on human health and economics. The metal nanoparticles will be used in much lower concentrations. Additionally, the enhanced interaction of metal nanoparticles with biological substances will lead to new applications and products.

Another component of the present invention provides a method for a surface plasmon resonance enhanced interaction of metal nanoparticles with biological substance in the presence of chemical agents. If chemical agents are near the electromagnetic fields of metal nanoparticles, their chemical activity can be changed and they can more effectively interact with the biological substance and with the metal nanoparticles. It was proven, without surface plasmon resonance, that a colloidal silver and hydrogen peroxide mixture is more effective at killing bacteria than these compounds working alone (Rami Pedahzur et al., “The interaction of silver ions and hydrogen peroxide in the inactivation of E. coli: a preliminary evaluation of a new long acting residual drinking water disinfectant”, Water Science and Technology Vol 31 No 5-6 pp 123-129 (1995)). In the presence of surface plasmon resonance and electromagnetic radiation, the disinfectant strength of the mixture will be higher; alternatively, the same disinfectant strength can be achieved with much lower concentrations of colloidal silver and hydrogen peroxide. The latter one is very important because of drinking water regulations regarding concentration limits of colloidal silver and hydrogen peroxide.

One of ordinary skill in the art would appreciate that the scope of the present invention includes a method of a surface plasmon resonance enhanced body treatment at a specific location. The method is as follows, but not limited to. Metal nanoparticles coated with a biorecognitive polymer are delivered to a specific place in the body and remain there.

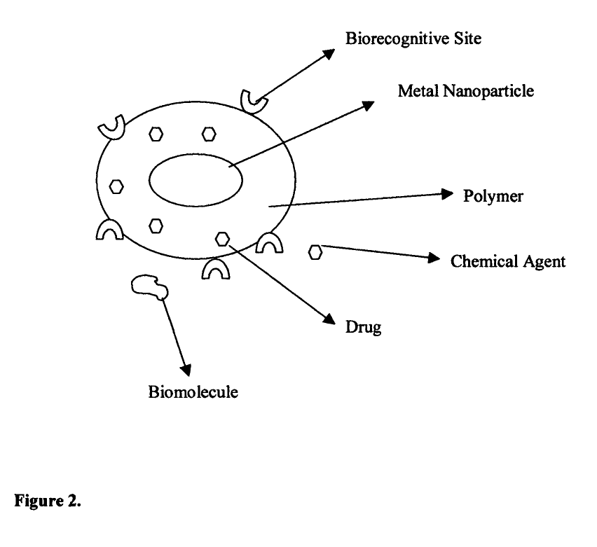

Generated surface plasmon resonance enhances the body treatment at this location in the body. If chemical agents or drugs are embedded into the polymer (FIG. 2) they can additionally enhance the body treatment. Electromagnetic radiation used in body treatment can be at single or plurality wavelengths, where one of the wavelengths is used for plasmon generation and the other wavelengths are used for interaction with the body and chemical agents/drugs. This method of localized body treatment can be applied, but not limited to, to cancer treatment and wound healing.

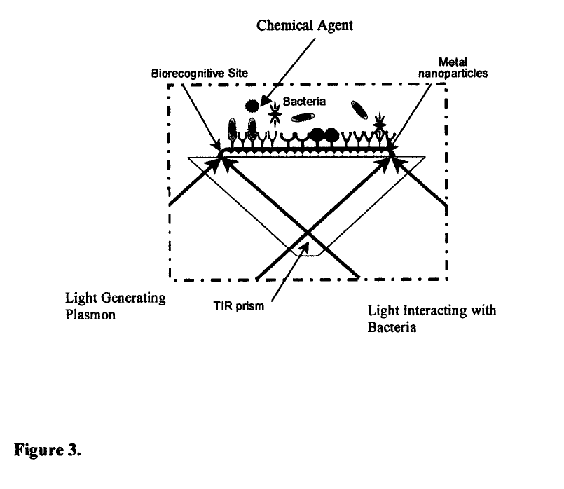

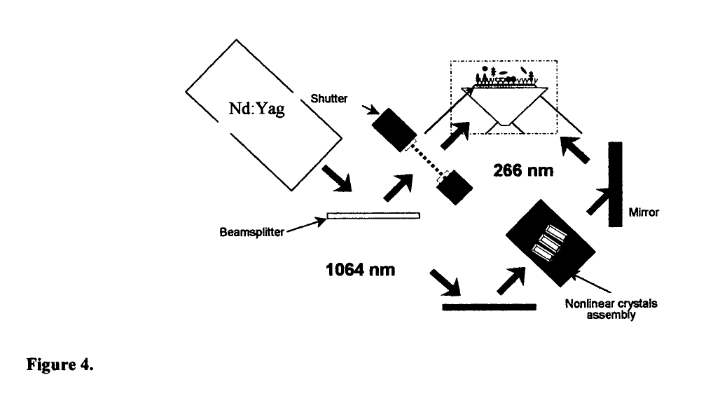

Another component of this invention is a surface plasmon resonance enhanced bacterial killing in a body, water, air, and in the other media. The metal nanoparticles used for bacterial killing can be in the form of colloids, spays, thin films (FIG. 3.), and others. Important for the surface plasmon resonance enhanced bacterial killing is a close proximity of bacteria to metal nanoparticles and to electromagnetic fields of surface plasmon resonance. The most enhanced bacterial killing effect occurs with non-coated metal nanoparticles, where bacteria are killed in direct contact with metal nanoparticle. However, coated metal particles have different advantage, biorecognitive sites on metal nanoparticles allowing for capturing and killing selected bacteria types. Additionally, in the invention is also described an alternative way for selection bacteria to be killed by illuminating bacteria at different wavelengths. It is known that bacteria have specific absorption spectra, which can be match with illuminating wavelengths to maximize effect of interactions of the illuminated bacteria with surface plasmon resonance. An example of an experimental set up for capture and killing bacteria in bioaerosol is showed on FIG. 4., where surface plasmon resonance is generated by a two-photon excitation and amino acids in bacteria are excited by 266 nm, a fourth harmonic of a Nd:Yag laser.

It will be understood by those skilled in the art that the present invention is a composition of novel and useful methods for a highly effective body treatment and bacterial killing in the presence of surface plasmon resonance, electromagnetic radiation, and chemical agents. The methods will be applied to the body externally for skin treatment, skin care and skin bacterial/viruses management, or internally for photodynamic body treatment and bacterial/viruses management. The surface plasmon resonance will enhance the following processes (but not limited to them): rejuvenating and regenerating tissue and nervous systems, removing skin wrinkles, preventing skin from aging and forming wrinkles, reducing skin odors, healing tissue injuries and skin burns, treating and preventing skin diseases, reducing joint inflammation and/or infection, reducing time and severity of body bacterial and viral infection, protecting/preventing/treating internally and externally the body against bacterial and viral infections, reducing effect of free radicals on body, treating and preventing tissue abnormalities, killing bacteria and viruses in body, killing bacteria and viruses in body environment.

The invention uses natural electromagnetic sources such as the sun (daylight) and bioluminescence as or specially designed electromagnetic sources like CW/pulsed and polarized/non-polarized light sources like lamps, LEDs, single and/or multiwavelength lasers. Surface plasmon resonance can also be generated by other techniques like sonic waves or electrical technologies (but not limited to them). The electromagnetic source has multiple roles in this invention, generating surface plasmon resonance and interacting with biological substances and chemical agents.

Sizes of the metal particles can vary from nanometers to micrometers and sizes are designed for best surface plasmon resonance interaction with biological substances and chemical agents. The colloidal metals for the use with biological substances and chemical agents will be dissolved in inorganic or organic liquids, ointments, collagens, sprays (but not limited to them).

Quadratic dependence of surface plasmon resonance generation on intensity of electromagnetic radiation, nonlinear multiphoton excitation, and high multiphoton absorption cross sections of metal nanoparticles significantly enhance the surface plasmon resonance interaction with body, bacteria and chemical agents, and allow for selection NIR wavelengths in deep tissue treatment.

Broadband structured absorption spectra of the colloidal metals from UVA to VIS/NIR allow for better body treatment and bacterial management and may also revolutionize skin sun-tanning technology.

The invention is applied to bacterial/viral management in water lines and/or air heating/conditioning systems in dental offices, hospitals, buildings, homes, swimming pools, bathtubs, Jacuzzi, air plains, trains (but not limited to them).

The invention is also applicable to personal hygiene products, cosmetics, household and industrial disinfectants and cleaners, antibacterial products in food industry (but not limited to them).

What is claimed is:

1. A method and a composition of a plasmon enhanced body treatment and bacterial killing comprises of:
a) Surface plasmon resonance and a metal nanoparticle interacting with a nearby biological substance,
b) Surface plasmon resonance and a metal nanoparticle interacting with a nearby biological substance and with a nearby chemical agent,
c) Surface plasmon resonance and a metal nanoparticle interacting with a nearby biological substance in the presence of electromagnetic radiation,
d) Surface plasmon resonance and a metal nanoparticle interacting with a nearby biological substance and with a nearby chemical agent in the presence of electromagnetic radiation,
e) A source for generating surface plasmon resonance in the metal nanoparticle,
f) An electromagnetic radiation source.

2. The method of claim 1, wherein the biological substance is selected from a group consisting of a biomolecule, tissue, skin, cells, body organs, bacteria, virus, pathogen, biochemical warfare agent, human body, animal body.

3. The method of claim 1, wherein the chemical agent is an inorganic molecule, organic molecule, mixture of inorganic and organic molecules, drug.

4. The method of claim 1, wherein the chemical agent is hydrogen peroxide.

5. The method of claim 1, wherein the metal nanoparticle is a metal, metallic salt, electric conductor, electric superconductor, electric semiconductor.

6. The method of claim 5, wherein the metal is selected from a group consisting of silver, ruthenium, platinum, rhenium, rhodium, osmium, iridium, copper, zinc, nickel, chromium magnesium, iron, palladium, gold, titanium, titanium dioxide, silver nitrate, alkaline earth metal, gold, copper, silver oxide, silver ion.

7. The method of claim 1, wherein the metal nanoparticle is coated with a biorecognitive material, bioactive material, dielectric material, chemorecognitive material, chemical active material, polymer, environmentally sensitive polymer, polymer containing drug.

8. The method of claim 1, wherein the metal nanoparticle is not coated with material.

9. The method of claim 1, wherein the metal nanoparticle size is in a range of 1 nm to 20,000 nm in at least one of the dimensions.

10. The method of claim 1, wherein the metal nanoparticles is a thin film, colloid, fiber, metal island, nanowire.

11. The method of claim 1, wherein a distance of the surface plasmon resonance enhanced interaction is from the metal nanoparticles (0 nm) up to 10,000 nm.

12. The method of claim 1, wherein the electromagnetic radiation source is selected from a group consisting of a laser with single wavelength, laser with plurality wavelengths, laser diode, light emitted diode, lamp, bioluminescence, sunlight, chemiluminescence, electroluminescence, metal nanoparticle luminescence.

13. The method of claim 1, and 12, wherein the electromagnetic radiation source is a single wavelength source of polarized or unpolarized light with wavelength between 200 nm to 10,000 nm,

14. The method of claim 1, and 12, wherein the electromagnetic radiation source is plurality wavelengths source of polarized or unpolarized light with wavelengths between 200 nm to 10,000 nm.

15. The method of claim 1, wherein the surface plasmon resonance is generated by electromagnetic radiation in a single-photon mode of excitation, multi-photon mode of excitation.

16. The method of claim 1, wherein the body treatment is a joints treatment, tissue treatment, cosmetic treatment, cosmetic prevention, rejuvenating treatment, therapy treatment, bacterial disease treatment, antibacterial treatment, virus treatment, cancer treatment, biostimulation treatment, antiodor treatment, sun prevention treatment, sunburn treatment, skin burn treatment, wound treatment, antiinflammation treatment.

17. The method of claim 1, 7, and 8, wherein the body treatment is performed at a specific location in the body, where said the metal nanoparticle remains in the location for the body treatment.

18. The method of claim 1, wherein the surface plasmon resonance enhanced body treatment and bacterial management is additionally enhanced by the nearby presence of electromagnetic radiation, the chemical agent, electromagnetic radiation and the chemical agent.

19. The method of claim 1, wherein bacterial killing is applied to: air conditioning and heating system, air humidity control system, air ventilation system, disinfectant product, antiseptic product, water supply line, water container, septic tank, bathtub, whirlpool, Jacuzzi, swimming pool, dental waterlines, food technology, animal food technology, household cleaning product, kitchen product, product for pets, cosmetic product, hygiene product, medical bio-safety product, hair product, laundry product, textile material, pharmaceutical product for human, pharmaceutical product for animal, health supplement product, drinking water product, beverage product, paint product, biodefense product, furniture preserving product, art preserving product, sunburn protection product, sun-tanning technology.

20. The method of claim 1, wherein the source for generation surface plasmon resonance in the metal nanoparticle is electromagnetic radiation, sonic wave technologies, electrical technologies, magnetic technologies, radiation technologies.
